(19) United States
(12) Patent Application Publication
(10) Pub. No.: US 2026/0192129 A1
(43) Pub. Date: Jul. 9, 2026

(54) SYSTEM AND METHOD FOR CONTROLLING AGAINST OR TREATING OF TYMPANOSTOMY TUBE INFECTIONS

(71) Applicants: The General Hospital Corporation, Boston, MA (US); Boston Medical Center Corporation, Boston, MA (US)

(72) Inventors: Tianhong Dai, Malden, MA (US); Stephen I. Pelton, Boston, MA (US); R. Rox Anderson, Boston, MA (US); Sabharwal Vishakha, Brookline, MA (US)

(21) Appl. No.: 19/126,614

(22) PCT Filed: Nov. 6, 2023
(86) PCT No.: PCT/US2023/078805

§ 371 Date: May 1, 2025

Related U.S. Application Data
(60) Provisional application No. 63/382,399, filed on Nov. 4, 2022.

(51) Int. Cl. A61N 5/06

(52) U.S. Cl. CPC A61N 5/0624; A61N 5/0603; A61N 2005/0605; A61N 2005/063; A61N 2005/0651; A61N 2005/0663

(57) ABSTRACT

Aspects of the present disclosure provide a system for preventing or treating tympanostomy tube infections. The system includes an ear tube that is configured to be placed within an ear of a subject. The ear tube has a shaft that extends from a proximal end to a distal end, and a lumen extends through the shaft. The ear tube also includes a flange extending from the shaft at the proximal end and a flange extending from the shaft at the distal end. The system further includes an optical waveguide that is configured to deliver antimicrobial light to a surface area of the ear tube while placed within the ear of the subject. The optical waveguide includes an optical fiber that is configured to deliver the antimicrobial light and a fiber collimator that is configured to guide the antimicrobial light towards the surface area of the ear tube.

### CROSS-REFERENCE TO RELATED APPLICATIONS

This application is based on, claims priority to, and incorporates herein by reference for all purposes, U.S. Provisional Patent Application No. 63/382,399 filed on Nov. 4, 2022.

### STATEMENT REGARDING FEDERALLY SPONSORED RESEARCH

This invention was made with government support under R21 DC019958 awarded by the National Institutes of Health. The government has certain rights in the invention.

### BACKGROUND

Tympanostomy tube (TT) placement for otitis media with effusion is the most common surgical procedure performed in children. In the United States alone, over 600,000 TT placement procedures are performed on a yearly basis. Ten percent (10%) of children aged 0 to 3 have at least one set of TTs placed, and 4.6% require two or more TT sets. TT has been shown to significantly restore hearing, reduce effusion prevalence, mitigate the middle ear inflammation, and shorten the duration of recurrent otitis media episodes. However, tympanostomy tube otorrhea (TTO), which is defined as active drainage through an existing TT and caused by the presence of pathogenic bacteria in the middle ear, is the most common complication after TT placement. The mean incidence of TTO is reported to be 26% in observational studies and up to 83% with prospective surveillance. TTO are accompanied with foul odor, fever, pain and pyrexia, can lead to tube occlusion and discomfort, and as such reduce quality of life for child and family. Haemophilus influenzae (H. influenzae), Staphylococcus aureus (S. aureus), and Pseudomonas aeruginosa (P. aeruginosa) are the most common pathogens isolated in TTO cases. The current standard of care for TTO is the treatment with topical antibiotics. However, despite this treatment, TTO is often persistent or recurrent. Microbial biofilms on TTs are implicated to be the major cause of persistent or recurrent TTO. The microstructure of biofilms contributes to their lower permeability and susceptibility to antibiotics, making it extremely difficult to eradicate bacteria on TTs. Coating of antimicrobial materials on TTs and tubal impregnation with antimicrobial materials have been studied to prevent biofilm formation on TTs, but the outcomes are reported to vary significantly. Additionally, the increasing emergence of antibiotic-resistant bacteria has further complicated the treatment of TTO. Therefore, there is an increasing need for the development of new treatments for TTO.

### SUMMARY OF THE DISCLOSURE

The present disclosure addresses the aforementioned drawbacks by providing a system and method for controlling against or treating tympanostomy tube infections.

Some aspects of the present disclosure provide a system for preventing or treating tympanostomy tube infections. The system includes an ear tube that is configured to be placed within an ear of a subject. The ear tube has a shaft that extends from a proximal end to a distal end, and a lumen extends through the shaft. The ear tube also includes a flange extending from the shaft at the proximal end and a flange extending from the shaft at the distal end. The system further includes an optical waveguide that is configured to deliver antimicrobial light to a surface area of the ear tube while placed within the ear of the subject. The optical waveguide includes an optical fiber that is configured to deliver the antimicrobial light and a fiber collimator that is configured to guide the antimicrobial light towards the surface area of the ear tube.

Other aspects of the present disclosure provide a method for treating or preventing tympanostomy tube infections. The method includes inserting a tympanostomy tube treatment system into an ear of a subject. The treatment system includes a moldable earplug that is configured to rest in the ear of the subject and an optical waveguide that is configured to deliver antimicrobial light to a tympanostomy tube. The method further includes delivering the antimicrobial light through the optical waveguide to a surface of the tympanostomy tube for a treatment duration. The method further includes removing the tympanostomy tube treatment system from the ear of the subject.

Other aspects of the present disclosure provide an ear tube that is configured to be placed within a tympanic membrane of an ear of a subject. The ear tube includes a shaft that extends from a proximal end to a distal end and a lumen that extends through the shaft. The ear tube further includes a first flange extending annularly from the shaft at the proximal end and a second flange extending annularly from the shaft at the distal end. The ear tube is fabricated from a primary material that is sufficiently transparent to antimicrobial light and has a plurality of pockets dispersed throughout the primary material. Each of the plurality of pockets is filled with a secondary material that is configured to scatter the antimicrobial light toward a surface of the ear tube.

These are but a few, non-limiting examples of aspects of the present disclosures. Other features, aspects and implementation details will be described hereinafter.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1A illustrates an example system for controlling against or treating tympanostomy tube infections in accordance with some aspects of the present disclosure.

FIG. 1B illustrates a view of an example earplug for use with the system of FIG. 1A.

FIG. 1C illustrates a second view of the example earplug of FIG. 1B.

FIG. 1D illustrates another example earplug placed in the ear of a subject.

FIG. 2A shows a front view and a side view of an example ear tube for use in accordance with the present disclosure.

FIG. 2B shows several additional example ear tubes for use in accordance with the present disclosure.

FIG. 2C illustrates another example ear tube, which includes a second material, for use in accordance with the present disclosure.

FIG. 3 is a flowchart setting forth the steps of an example process for controlling against or treating tympanostomy tube infections.

FIG. 4 illustrates steps of an example process for molding ear tubes.

FIG. 5 illustrates prototype ear tubes in accordance with some aspects of the present disclosure.

FIG. 6A illustrates an example experimental setup for measuring antimicrobial light transmission in a first example ear tube material.

FIG. 6B shows experimental results generated by the experimental setup illustrated in FIG. 6A.

FIG. 7A illustrates an example experimental setup for measuring antimicrobial light transmission in a second example ear tube material.

FIG. 7B shows experimental results generated by the experimental setup illustrated in FIG. 7A.

FIG. 8A illustrates an example experimental setup for measuring antimicrobial light transmission in a third example ear tube material.

FIG. 8B shows experimental results generated by the experimental setup illustrated in FIG. 8A.

FIG. 9 shows experimental results from a first experimental setup measuring the heating effect caused by antimicrobial light absorption of the ear tubes.

FIG. 10 shows experimental results from a second experimental setup measuring the heating effect caused by antimicrobial light absorption of the ear tubes.

FIG. 11 shows experimental results measuring the efficacy of antimicrobial light treating H. influenzae in a first experimental setup.

FIG. 12 shows experimental results measuring the efficacy of antimicrobial light treating H. influenzae in a second experimental setup.

FIG. 13 shows experimental results testing the biofilm formation of otopathogens on ear tubes.

FIG. 14 shows experimental results testing the efficacy of antimicrobial light treating H. influenzae, P. aeruginosa, and S. aureus on ear tubes.

### DETAILED DESCRIPTION

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The present disclosure provides systems and methods to control against and/or treat infections related to the use of tympanostomy tube. In some aspects of the disclosure, an optical tympanostomy tube (Optical-TT) system may be provided, which can be coupled to an excitation source. The excitation source can emit antimicrobial light (for example, antimicrobial blue light (aBL)). The optical TT is designed to receive the antimicrobial light from the source, direct the antimicrobial light both through the optical TT and to emit the antimicrobial light from the body of the optical TT in a manner designed to control against and/or treat infections in or proximate to the ear where the optical TT has been deployed.

The current standard of care for TTO is the treatment with topical antibiotics. However, despite this treatment, TTO is often persistent or recurrent. The present disclosure recognizes that microbial biofilms on TTs are implicated to be a major cause of persistent or recurrent TTO. The microstructure of biofilms contributes to its lower permeability and susceptibility to antibiotics, making it extremely difficult to eradicate bacteria on TTs. Coating of antimicrobial materials on TTs and tubal impregnation with antimicrobial materials have been studied to prevent biofilm formation on TTs, but the outcomes are reported to vary significantly. Additionally, the increasing emergence of antibiotic-resistant bacteria has further complicated the treatment of TTO. Therefore, there is an increasing need for the development of new treatments for TTO.

As stated, the excitation source may deliver antimicrobial light, which may have a mechanism of action of photoexcitation of naturally occurring endogenous photosensitizing chromophores (primarily iron-free porphyrins) in bacteria and the subsequent production of cytotoxic oxidative species. The present disclosure seeks to harness advantages of antimicrobial light over traditional antibiotics, such as rapid action and equal killing effectiveness irrespective of the antibiotic resistance profiles of bacteria. Bacteria may also be less able to develop resistance to antimicrobial light than to antibiotics, due to the multitarget mode of action of antimicrobial light. In comparison with traditional antimicrobial photodynamic therapy, which combines visible light and exogenous photosensitizers, antimicrobial light is more appealing in that it kills bacteria without the involvement of exogenous photosensitizers.

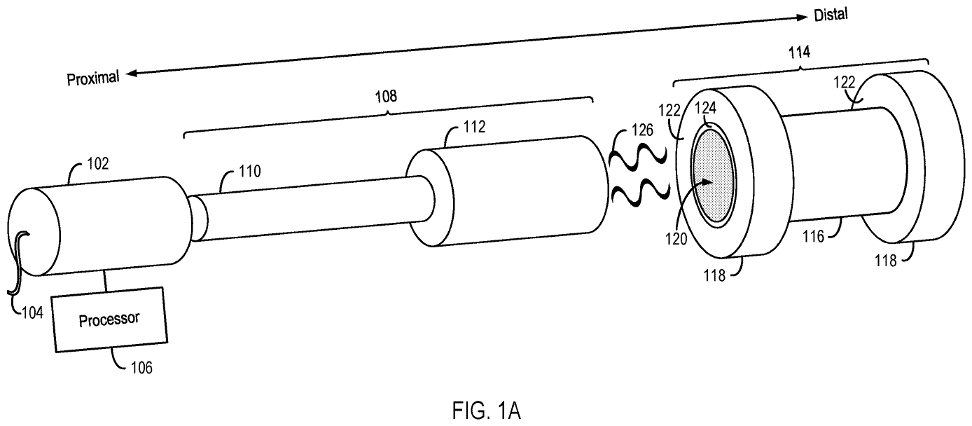

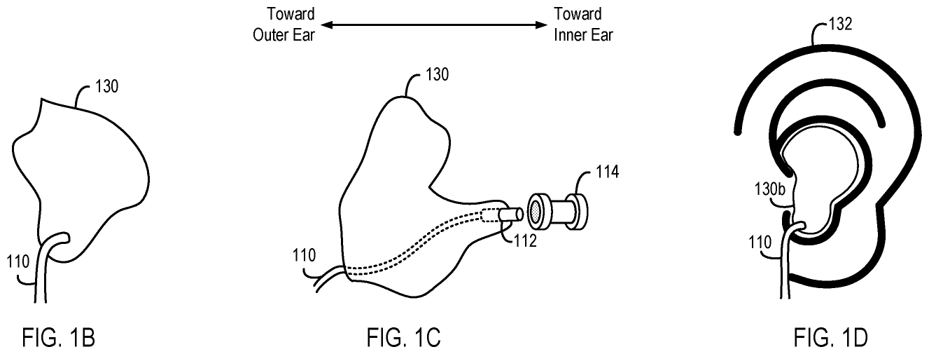

When developing the systems and methods of the present disclosure, a variety of research and studies were conducted. In one study, we demonstrated that aBL was effective in killing H. influenzae, S. aureus, and P. aeruginosa in in vitro biofilms. Furthermore, we found that no statistically significant viability loss of human cells occurred under the therapeutic exposures of aBL for killing bacteria in biofilm cultures. A schematic of one example of an Optical-TT system developed as described herein is illustrated in FIGS. 1A-1D. The system may include a light or excitation source 102, an optical waveguide 108, and a tympanostomy or ear tube 114 designed to be coupled to the waveguide 108 and light source 102. The system may also include an earplug 130.

The light source 102 can be configured to produce or emit antimicrobial light 126. For example, the antimicrobial light 126 may be antimicrobial blue light and have a wavelength between 400 nm and 470 nm or between 405 nm and 420 nm. The light source 102 may include a power source 104, such as a battery or standard power outlet. In some configurations the light source 102 may be portable. In some configurations, the light source 102 may also include or be in electrical communication with a processor 106 that can control the emission of the light source. For example, the processor 106 may control the timing (e.g., start and stop time, repetition time), irradiance, wavelength, energy, power, light exposure, emission pattern, etc. of the light emission. The light source 102 may include a laser, a diode laser, a femtosecond laser, a light-emitting diode, another suitable light source, or a combination thereof.

The optical waveguide 108 may be located between the light source 102 and the ear tube 114. The optical waveguide 108 can be configured to deliver or direct the antimicrobial light 126 from the light source 102 to the tube 114. The optical waveguide 108 may include an optical fiber 110 at its proximal end and a collimator 112 at its distal end. For example, the optical fiber 110 can be a multi-mode optical fiber that has a refractive index chosen based on the size of the fiber and the wavelength of antimicrobial light 126 used. In use, the optical fiber 110 can direct the antimicrobial light 126 from the light source towards the tube 114, and the collimator 112 can align the antimicrobial light 126 to efficiently irradiate the tube 114. In this way, the collimator 112 can direct light toward the outside or proximal end of the tube 114 and through the tube material. In use, optical waveguide 108 may be placed proximate to the tube 114. In some configurations, the collimator 112 of the optical waveguide 108 may be spaced from the tube 114 to avoid transmitting bacteria or other pathogens from the device to the tube 114. In other configurations, the collimator 112 may be configured to touch the tube 114. For example, the optical waveguide 108 may be sterilized and placed in contact with the tube 114 to efficiently transmit antimicrobial light 126.

As illustrated in FIGS. 1B-1D, to facilitate easy placement of the optical waveguide 108, it may be partially embedded within or configured to engage with a moldable or flexible earplug 130. In some configurations, the earplug 130 may be custom molded to fit into a specific patient's outer ear and ear canal. The earplug 130 can be configured to comfortably rest within the patient ear canal and align the collimator 112 with an outer or proximal end of the tube 114. For example, one example earplug 130 is provided in FIGS. 1B-1C, and a second example earplug 130b is provided in FIG. 1D.

The use of an earplug 130 provides patients with a comfortable treatment and consistent placement from one treatment to the next. As FIG. 1C shows, the optical fiber 110 may enter into the earplug 130 on one side or face of the earplug 130 that faces the outer ear. The optical fiber 110 may connect to the collimator 112 within the earplug 130 and exit the earplug 130 on another end or face that faces the inner ear. In this way, a distal end of the optical fiber 110 and a proximal end of the collimator 112 may be embedded in or fixed in the earplug 130. The position of the collimator 112 within the earplug 130 may be chosen in order to align the collimator with the tube 114 when the earplug 130 is placed into the ear 132 of the subject. In use, the earplug 130 can be placed into the ear 132 of the subject when treatment is required, as shown in FIG. 1D, and removed after a treatment session is completed. This can be repeated as desired to achieve a desired clinical result. Advantageously, the design and consistency provided by the earplug 130 allows the device to be used during a clinic visit or by a user or patient at home.

The ear tube 114, which may be referred to as a tympanostomy tube (TT) or an optical tympanostomy tube (OTT) is configured to be inserted into an ear 132 of a patient or subject. In use, the tube can be surgically placed in the patient's tympanic membrane or eardrum to create a channel or opening between the ear canal and the tympanic cavity.

Referring again to FIG. 1A, the tube may be sized to fit into the tympanic membrane or tympanic cavity of the particular subject. The tube 114 may have a main shaft 116 extending from a proximal end to a distal end. The shaft 116 can have a wall 124 that forms a lumen 120 or canal extending through the shaft 116 from the proximal end to the distal end of the tube 114. The tube 114 may have a pair of flanges 118 or ridges that extend annularly from the shaft 116 at the proximal end and the distal end, respectively. The flanges 118 have an outer wall 122 of a desired thickness. In this way, the tube 114 may have a thicker width or diameter at its proximal and distal ends than it does along the main shaft, forming a dumbbell-like or collar button-like shape.

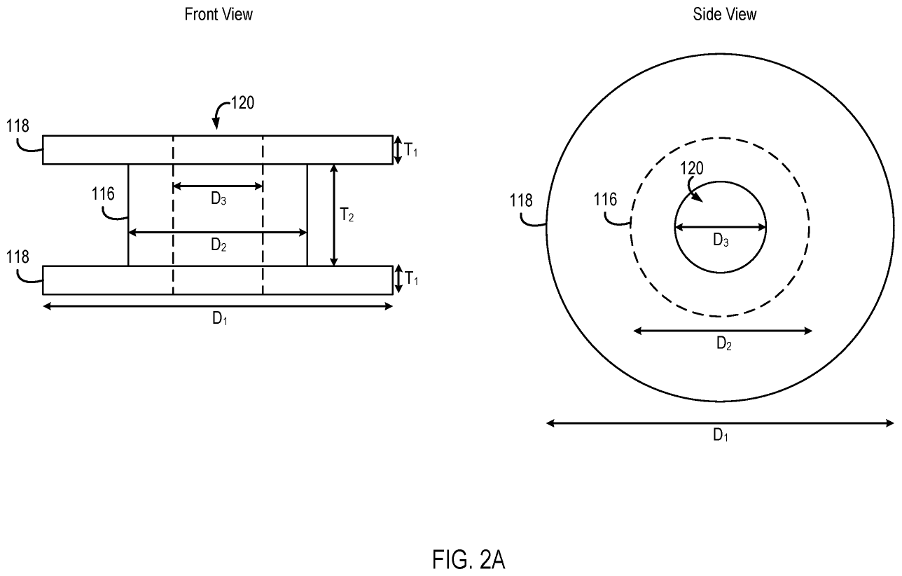

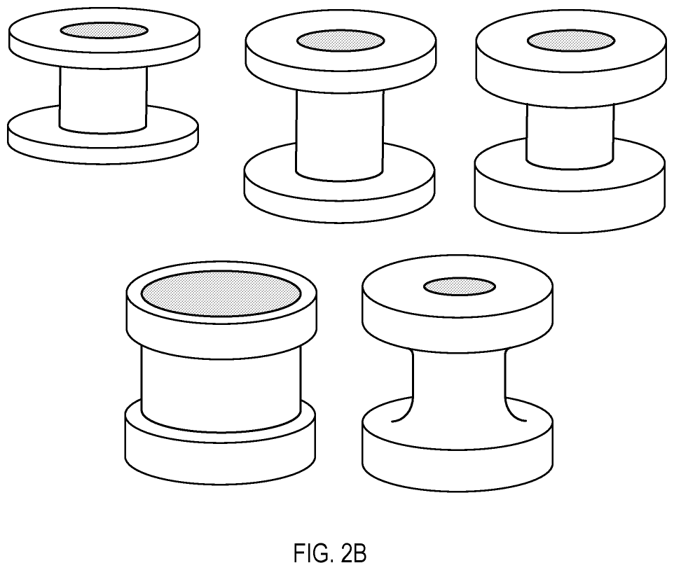

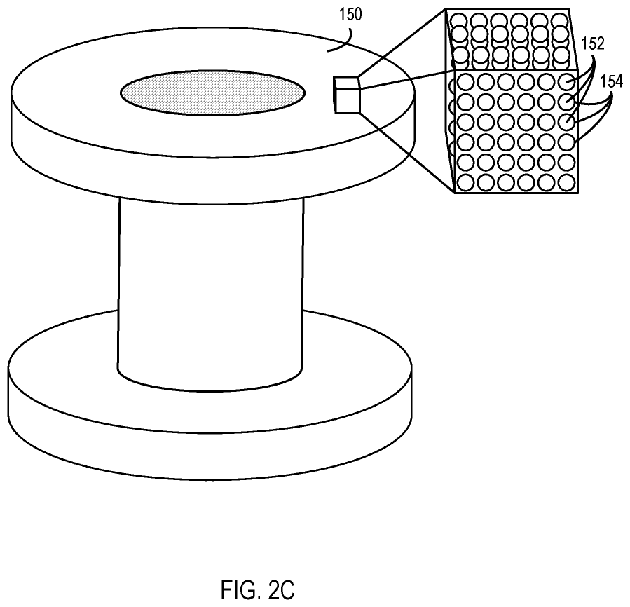

One non-limiting example of dimensions of the tube 114 is shown in FIG. 2A. The tube has a width or diameter, D₁, of the flanges 118 and a width or diameter, D₂, of the main shaft 116. The tube 114 also has a width or diameter, D₃, of the lumen 120. In general, D₁≥D₂≥D₃. The tube 114 can further be described by a length or thickness, T₁ of each of the flanges 118 and a length or thickness, T₂, of the main shaft 116. The overall length of the tube can be described by 2*T₁+T₂. As two non-limiting examples, the tube 114 may be manufactured with the following dimensions:

| | D₁ | | D₂ | | D₃ | | T₁ | | T₂ | |
|---|---|---|---|---|---|---|---|---|---|---|
| Example 1 | 3 | mm | 1.7 | mm | 1.1 | mm | 0.3 | mm | 1.5 | mm |
| Example 2 | 2.9 | mm | 1.7 | mm | 1.2 | mm | 0.35 | mm | 1.4 | mm |

The particular dimensions of the tube 114 may be determined based on the patient or the application. Several example tubes are shown in FIG. 2B with varying proportions and dimensions.

The tube 114 can be configured from a primary material that is biocompatible. For example, the material may include an elastomer, a polymeric organosilicon compounds or silicone, a 3D printing material, or other flexible material. The material may preferably be flexible. It may also have a specifically-selected optical absorption over a spectrum of antimicrobial light 126. For example, the tube 114 can be designed to transmit antimicrobial light 126 through the tube 114, but also disperse the antimicrobial light 126 so that the surface of the tube 114 can be irradiated with antimicrobial light 126 in use.

As a non-limiting example, the material may be transparent or sufficiently transparent to antimicrobial light. For example, the material may transmit at least 90% of sunlight and at least 70% of ultraviolet irradiation. As another non-limiting example, the material may have a maximum limit on the light loss per mm of material. For example, the material may have a light loss limited to 0.0001/mm, 0.001/mm, 0.01/mm, 0.1/mm, 0.2/mm, and so forth.

In some configurations, the tube 114 may be manufactured using 3D printing or polymer molding techniques. As one non-limiting example, the tube 114 may be manufactured (e.g., molded) from a silicone, a fluoroplastic, a thermoplastic polymer (e.g., polymethylmethacrylate (PMAA)), an elastomer (e.g., polydimethylsiloxane (PDMS)), or a polymethyl methacrylate acrylic (e.g., Plexiglas® V825). The material may provide flexibility of the tube. For example, PDMS belongs to a group of polymeric organosilicon compounds that are commonly referred to as silicones and is an FDA approved material widely used in fabrication of biomedical devices. The flexibility of PDMS allows for easy insertion of the tube without any damage to tympanic membranes. It also possesses low optical absorption over the entire visible spectrum, rendering it an ideal material for this application. As another non-limiting example, the tube 114 may be fabricated using 3D printing techniques (e.g., PolyJet™) using a 3D printing material. As non-limiting examples, the 3D printing material may include a 3D printing material that simulates PMMA or acrylic (e.g., VeroClear™), a low moisture absorption ABS-like material (e.g., ABS-Like Translucent/Clear™), or another suitable 3D printing material.

As illustrated in FIG. 2C, in some configurations, the tube 114 may include a second material 152 within the primary tube material (e.g., PDMS, silicone, etc.) 150. The second material 152 may be configured to scatter the incident antimicrobial light 126 such that the tube 114 scatters some of the antimicrobial light and disperses the light to the surface of the tube 114 to achieve a more uniform or more complete coverage of the surface of the tube 114. The second material 114 may be dispersed throughout the primary material, uniformly, randomly, or otherwise. For example, pockets 154 or holes may be formed throughout the main body of the tube within the primary material. The pockets 154 may be filled or partially filled with the second material 152.

The second material may include a solid, liquid, gas, or combination thereof. For example, the second material 152 may include gas bubbles or microbubbles filled with a gas, such as air, nitrogen, carbon dioxide, etc. In other configurations the second material 152 may include solid particles or nanoparticles. For example, the solid material may include organic or inorganic, such as metal, polymers, carbon nanotubes, liposomes, gold nanoparticles, silver nanoparticles etc. The second material 152 may also include a liquid or gel. For example, small holes may be formed in the tube material that may be filled with a liquid or gel. As one non-limiting example, the tube may include water drops distributed throughout the walls of the tube. The bubbles, solid particles, or drops may be evenly, randomly, or otherwise distributed throughout the body of the tube.

In some configurations, the tube 114 may additionally be impregnated with another anti-microbial material. For example, the tube may be impregnated with silver oxide, which may suppress postoperative infection.

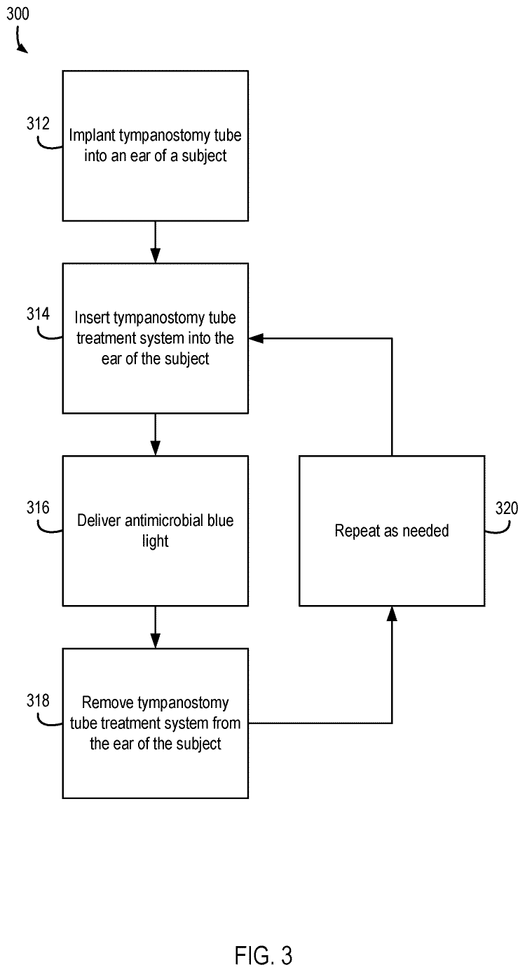

FIG. 3 shows a flowchart illustrating a process 300 for treating a tympanostomy tube with antimicrobial light. The tympanostomy tube may be surgically placed, as indicated in 312. The tube may be placed in the tympanic membrane of one or both ears of a subject or patient. This creates a channel between the ear canal and the tympanic cavity to prevent pressure buildup within the ear. Once the tube is in place, treatment can be applied intermittently. The treatment system can be placed within the ear of the subject as indicated in 314. Placing the treatment system may include inserting the earplug into the ear canal of the subject. The earplug may be custom fit to the ear or moldable to create a snug fit within the ear. Placing the earplug can align the collimator of the waveguide with an outer end of the tympanostomy tube. As indicated in 316, the light source can be used to emit antimicrobial light through the waveguide and through the tympanostomy tube, irradiating the surface of the tympanostomy tube. The antimicrobial light may be configured with a desired timing (duration, start time, stop time, pulse pattern), wavelength, and power. As non-limiting examples, the irradiation duration may be set as 1 min, 5 min, 10 min, 20 min, 30 min, 40 min, 50 min, 60 min, or the like. The irradiation may also have a pulse pattern or duty cycle, such that the irradiation is intermittently turned on and off. For example, the antimicrobial light may be delivered for a set duration while repeatedly pulsed on for 1 second and off for 1 second. Other pulse patterns may be used. Such duty cycle may reduce potential side effects to surrounding tissues.

After the treatment session, the treatment system can be removed from the patient's ear, as indicated in 318. The treatment process can be repeated, as in 320, as desired to achieve a target clinical effect. As a non-limiting example, the treatment may be applied for a 10-minute treatment session, one-three times per day for one week, or until the ear tube is removed or the infection is eradicated. As another non-limiting example, treatment may be applied 1-2 times per month to prevent infection. The power may be set such that the irradiance on the surface of the tube is ≤100 mW/cm², for example. For example, the power may be set to achieve irradiance of 1 mW/cm², 5 mW/cm², 10 mW/cm², 20 mW/cm², 30 mW/cm², 40 mW/cm², 50 mW/cm², 75 mW/cm², 100 mW/cm², or the like. Other treatment parameters may also be used.

In some configurations, the treatment parameters (e.g., irradiation duration, pulse pattern, irradiance, and so on) may depend on the specific clinical situation. For example, if an infection is detected, treatment may be applied more frequently, with higher power, or for longer durations, for example. If infection is not detected, lower treatment parameters may be used to prevent infection from occurring. In some configurations, the particular bacteria may determine treatment parameters. For example, the infection may be cultured to test for particular strains or species of bacteria. If the bacteria detected are more resilient to antimicrobial light treatment, the treatment parameters may be increased.

In some configurations, the process 300 may additionally include treating the subject's ear with topical antibiotics or other medicament. As a non-limiting example, ciprofloxacin 0.3%/dexamethasone 0.1% otic drops may be applied into the ear canal prior to or after antimicrobial light treatment. The antimicrobial light therapy and antibiotics may have a synergistic effect to treat the bacterial infection more effectively.

### EXAMPLES

### Example 1

Preliminary studies have shown that aBL is effective in killing H. influenzae, S. aureus, and P. aeruginosa in experimental biofilms. Furthermore, we found that no statistically significant viability loss of human cells occurred under the therapeutic exposures of aBL for killing bacteria in biofilms. We found that using an irradiance of the tube ≤100 mW/cm² does not produce thermal effect on biological tissues.

### Example 2

To form biofilms, 200-μL H. influenzae suspensions containing 106 colony forming units (CFU)/ml in brain heart infusion (BHI) broth were inoculated in 96-well microtiter plates and incubated at 37° C. for 24 h. After incubation, biofilms were exposed to aBL at increasing light exposures. When an exposure of 162 J/cm² aBL had been delivered (45 min irradiation at an irradiance of 60 mW/cm²; light exposure (J/cm²)=irradiance (W/cm²)×irradiation time(s)), 4.75-log₁₀ (99.998%) reduction of CFU in biofilms was achieved.

Additionally, we demonstrated that, at a single aBL exposure of 216 J/cm² (60 min irradiation at an irradiance of 60 mW/cm²), 7.15-log₁₀ (>99.99999%) and 1.58-log₁₀ (97.4%) CFU reductions were obtained in biofilms formed by multidrug-resistant clinical isolates of P. aeruginosa and S. aureus, respectively, in 96-well microtiter plates. We also found that the efficacy of aBL in killing S. aureus biofilm could be significantly improved (increased by ~2-log₁₀ CFU) by a preexposure of the biofilm to 460 nm wavelength light, which can photolyze staphyloxanthin, an antioxidant distributed in S. aureus.

No statistically significant viability loss of human cells was observed under therapeutic aBL exposures for killing TTO pathogens in biofilms. The human keratinocyte cell line HaCaT was studied as the representative human cell type. Suspensions of HaCaT cells (10⁶ cells/ml) in 35 mm petri dishes were exposed to aBL at varying light exposures. Annexin V and Propidium Iodide (PI) staining were used for flow cytometry to determine viable, apoptotic and necrotic cells. Compared to untreated cells, the viability of aBL-treated cells was reduced by approximately 5% and 7% at aBL exposures of 108 J/cm² and 216 J/cm², respectively. However, the differences were not statistically significant (P >0.05), indicating that, under the therapeutic exposures of aBL for killing TTO pathogens in biofilms, aBL only exhibited minimal or modest photo-cytotoxicity to normal human cells.

### Example 3

To determine the efficacy of Optical-TT in prevention and eradication of experimental biofilms on TTs in vitro, representative causative bacteria of TTO were studied. Experimental biofilms were formed directly on TTs. The bacterial species and strains included: H. influenzae: ATCC 62065, ATCC 33391, and 10 recent clinical isolates; S. aureus: ATCC 29213, ATCC 83254 and 10 recent clinical isolates; and P. aeruginosa: ATCC 19142, PA01 and 10 recent clinical isolates. The ATTC reference strains selected were those frequently used by previous studies of TTO and were demonstrated biofilm formers. The clinical isolates were obtained recently from patients with TTO due to chronic suppurative otitis media treated at Boston Medical Center and were selected according to the clinical importance of their antibiotic resistance profiles. The antibiotic resistance profiles of bacteria were determined using minimum inhibitory concentrations (MICs).

### Example 4: Fabrication of Antimicrobial Blue Light Actable Optical Tympanostomy Tube

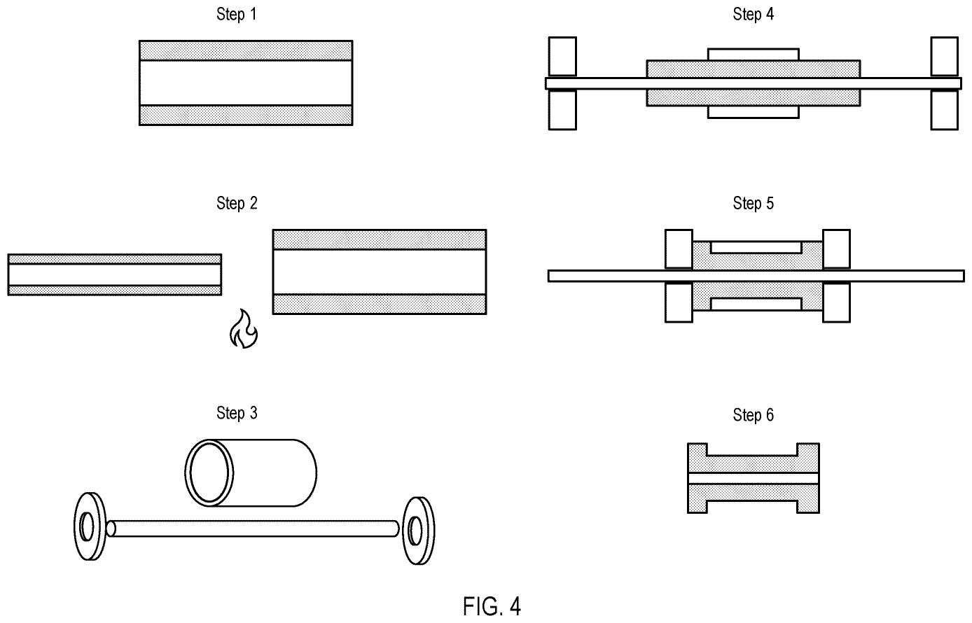

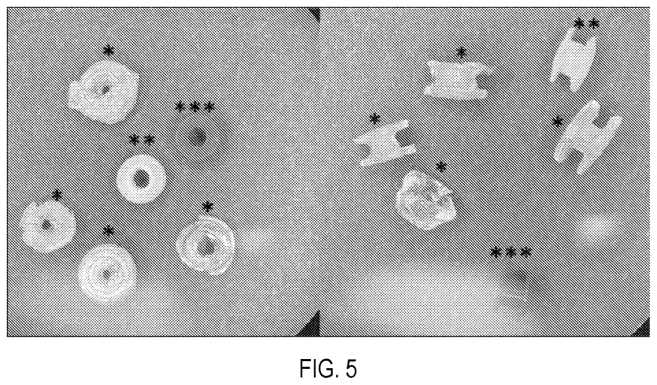

We aimed to identify the optical materials that are suitable for the massive fabrication of Optical-TTs and are also cost-effective. It is required that the materials be highly transparent to aBL (405 nm) with low absorption. We first tested thermoplastic polymers (polymethylmethacrylate; PMMA) and elastomers (polydimethylsiloxane; PDMS), both are widely used materials for fabricating medical devices. PMMA has high transmission in a broad light spectrum (>92% of sunlight and up to 73.5% of ultraviolet irradiation). Additionally, PMMA is feasible for the injection molding process to fabricate the Optical-TTs, allowing a large amount of production of TTs. At the stage of building prototypes for preliminary testing, we initially fabricated the Optical-TTs using PMMA with a manual method illustrated in FIG. 4. The Optical-TTs were fabricated in a ‘collar button’ shape, a common shape of commercial TTs, as shown in FIGS. 1A, 2A, 2B, and 4. However, we found later that the process of manual fabrication was challenging, since it frequently resulted in the asymmetry of the TTs and also surface irregularities, which could compromise light transmission and increase biofilm adherence. For example, FIG. 5 shows example fabricated optical-TTs. The TTs include manually fabricated using PMMA (*), commercial collar button TTs using fluoroplastic (**), and commercial collar button TTs using silicone (***).

Therefore, we opted to build additional TTs using 3D printing. Many different 3D printing materials may be used. In this experiment, we used a strong, durable, low moisture absorption ABS-like material (e.g., ABS-Like Translucent/Clear™) and a rigid transparent 3D printing material that simulates PMMA or acrylic (e.g., VeroClear™). Both materials are transparent photopolymers and possess similar mechanical and optical properties to those of PMMA. The prototype TTs were 3D printed using jetting photopolymer 3D printing technology (e.g., PolyJet™). We also produced molded TTs using a polymethyl methacrylate acrylic (e.g., Plexiglas® V825) with a molding injection and extrusion technique.

We first measured the transmittance of aBL in bulk samples. aBL was delivered by using a 405 nm diode laser (e.g., #19-478 available from Edmund Optics), and the transmittance of aBL was measured by using a power/energy meter (e.g., PM100D available from Thorlabs).

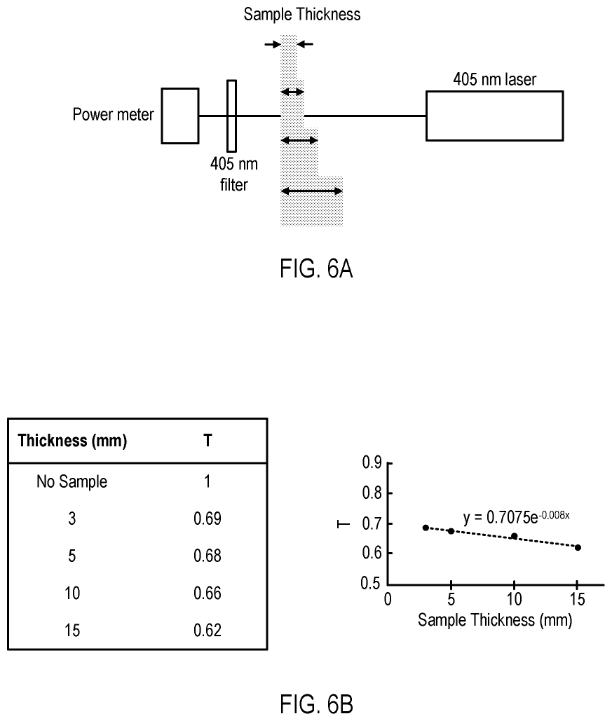

As demonstrated in FIG. 6A, to measure the transmittance of aBL in VeroClear™, the samples were polished on the surface to improve the light collection by the power meter. We observed ~29% light loss between the two facets. The Fresnel loss was ~10%, and the rest of the light loss was attributed to surface roughness. The surface quality of all tested samples was nearly identical. The transmittance measurements were correlated with the sample thickness using an exponential fitting. The light loss in VeroClear™ is 0.008/mm (or 0.035 dB/cm), as shown in FIG. 6B.

For ABS-Like Translucent/Clear™, we noticed that it is highly light-diffusing. Under a microscope, we observed microcracks or tiny surface irregularities all over the surface. We tried to eliminate the surface irregularities by polishing the sample surface. However, we did not observe any improvement. Interestingly, samples became transparent when they were wet, indicating that it was the surface irregularities that caused light scattering, not the optical properties of the material.

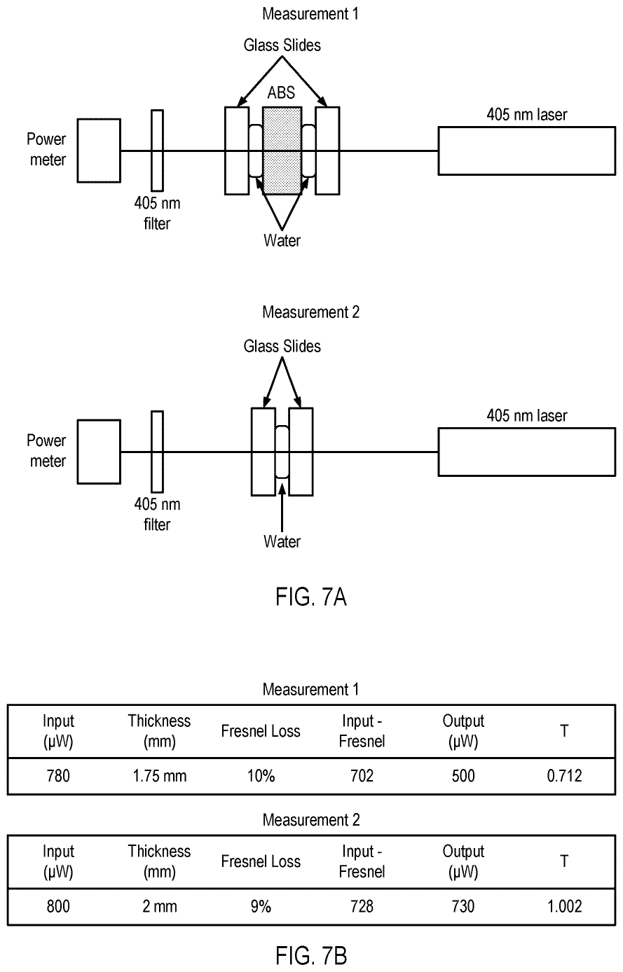

As demonstrated in FIG. 7A, to measure the transmittance of aBL in ABS-Like Translucent/Clear™, samples were placed between a pair of glass slides. A drop of water was added to the sample surfaces to mitigate the light reflection and reduce the light loss caused by the surface. Transmittance measurement showed that the light loss in ABS-Like Translucent/Clear™ is 0.194/mm (or 0.84 dB/cm), as shown in FIG. 7B.

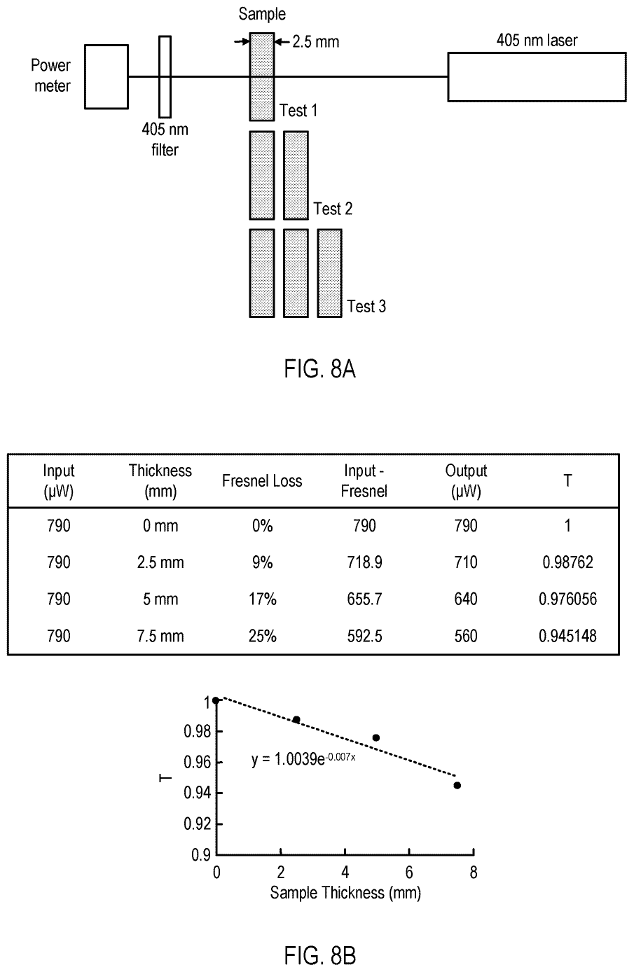

To measure the transmittance of aBL in Plexiglas® V825, we performed three tests using 1, 2, and 3 slabs, respectively, as demonstrated in FIG. 8A. The thickness of each slab was 2.5 mm. The slab surfaces were polished before the experiment. The transmittance measurements were correlated with material thickness using an exponential curve. The Fresnel loss (light loss) for Plexiglas® V825 is 0.007/mm (or 0.030 dB/cm), as shown in FIG. 8B.

Based on the measurements of aBL transmittance above, we selected VeroClear™ material for building the Optical-TT prototype via 3D printing. As the standard dimensions (e.g., thickness) of TTs were so small (~0.05 mm) that they are below the printable limit of the PolyJet™ 3D printer we used, we decided to test the Optical-TTs with increased dimensions.

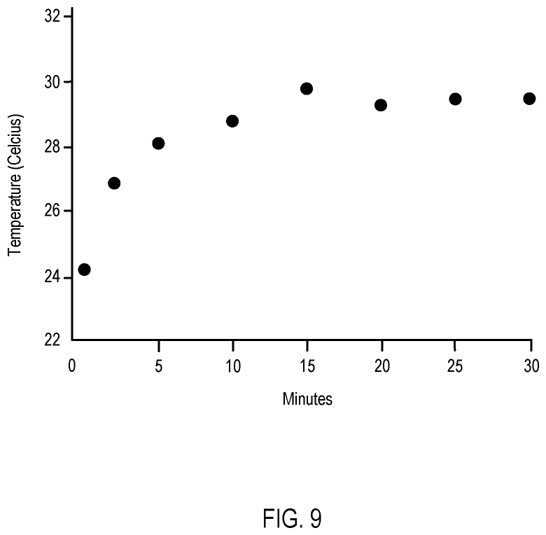

Next, we determined whether there was a heating effect due to aBL-absorption by the Optical-TTs. Overheating of the Optical-TT may cause thermal damage to the host tissue (i.e., tympanic membrane). We first measured the temperature of the Optical-TT using a thermos-couple (e.g., Traceable™ Total-Range Thermometer available from Fisherbrand™). The Optical-TT was illuminated using a single 405 nm light-emitting diode (LED) (e.g., M405L3 available from Thorlabs). The LED was positioned 4 cm above the outer facet of the Optical-TT, with the irradiance on the outer facet of the Optical-TT set to 60 mW/cm², as determined by a power meter (e.g., PM100D available from Thorlabs). The thermocouple sensor was in contact with the inner facet of the Optical-TT. After 15 min of aBL exposure (54 J/cm²), the temperature of the Optical-TT increased from 24.2° C. at 0 min to 29.8° C. at 15 min, a 5.6° C. increase, as shown in FIG. 9.

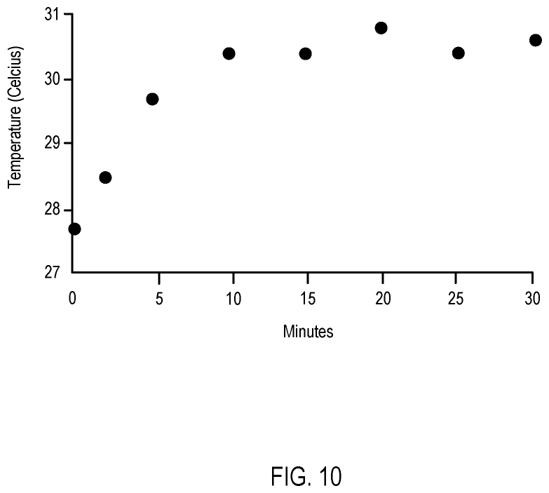

To avoid the effect of light absorption by the thermocouple sensor on the temperature measurement, we used aluminum foil to wrap up the senor. We observed an increase of temperature from 27.7° C. at 0 min to 30.8° C. at 20 min, a 3.1° C. increase after an aBL exposure of 72 J/cm², as shown in FIG. 10.

We also measured the temperature of the Optical-TT by using a forward-looking infrared (FLIR) thermal imaging camera (e.g., FLIR TG167). The FLIR camera was fixed using a holder, and the Optical-TT was placed on a polystyrene surface. The Optical-TT was illuminated using a 405 nm LED (e.g., M405L3 available from Thorla). The LED was positioned 4 cm above the outer facet surface of the Optical-TT, with the irradiance set to 60 mW/cm².

### Example 5: Investigation of aBL Efficacy in Killing Otopathogens In Vitro

We investigated the efficacy of aBL in killing multiple clinical isolates of H. influenzae, an important otopathogens species, in either planktonic cultures or biofilms in vitro.

aBL was delivered using a single LED with a peak emission of 405 nm and a full width at half-maximum (FWHM) of 25 nm (e.g., M405L3 available from Thorlabs). The LED was positioned 4 cm above the bacterial culture surface, and the irradiance of light was set to 60 mW/cm².

Five H. influenzae clinical isolates strains (NTHi RD118, NTHi 1003 wt, NTHi BCH1, NTHi 375 wt, and NTHi 486 wt), which were collected from TTO patients at Boston Medical Center, were studied. Additionally, a ATCC reference strain (ATCC 10211) was also included. For all experiments, the bacteria were cultured on Chocolate agar and in Brain Heart Infusion (BHI) broth supplemented with 2 mg/mL nicotinamide adenine dinucleotide and 10 mg/mL hemin, and incubated at 37° C., for 18-24 h under ~5% CO2.

Overnight bacterial suspensions were centrifuged at 4,000×g for 10 min, washed twice in phosphate buffered saline (PBS), resuspended in fresh PBS. The inocula of bacterial suspensions were set to 1-2×108 colony forming units (CFU)/mL by adjusting the optical density (OD=0.1) of the suspensions by a spectrophotometer (λ=600 nm; optical path=1 cm). Then, bacterial suspensions in 1 mL were transferred to the wells of 48-well plates and exposed to aBL at increasing radiant exposures of 0, 36, 72, 108, 144, and 180 J/cm², reflecting 0, 10, 20, 30, 40, and 50 min irradiation time, respectively. After each radiant exposure of aBL, aliquots were taken, serially diluted, and plated on Chocolate agar. Bacterial viability was quantified by CFU counting, and the data was presented in log₁₀ CFU/mL reduction. All the experiments were performed in three independent replicates on 3 different days, respectively.

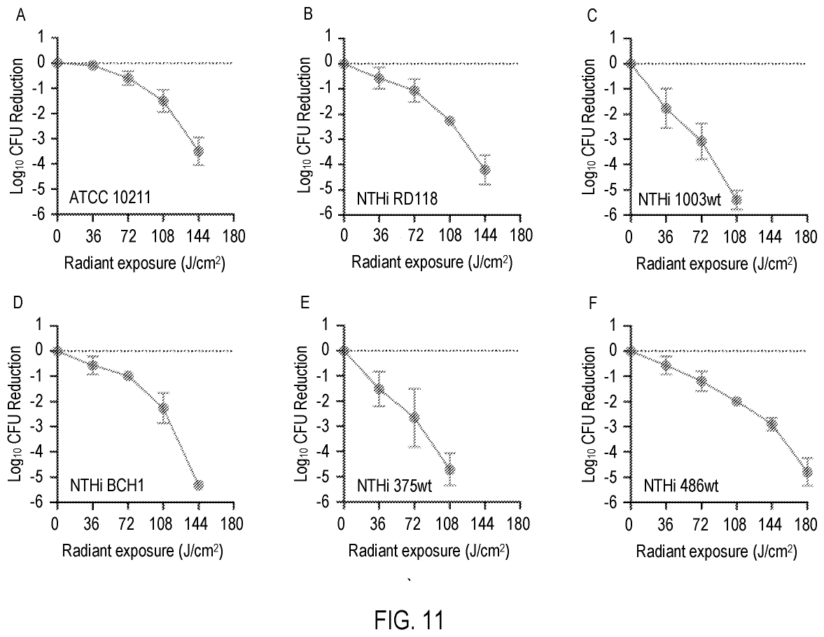

As shown in FIGS. 11A-F, all the strains exhibited high susceptibility to killing by aBL in planktonic cultures, with variations in susceptibility in different strains. NTHI 375 wt strain was the most sensitive to aBL, reaching 4.7-log₁₀ CFU reduction after an aBL exposure of 108 J/cm², followed by NTHi 1003 wt, with 5.4-log₁₀ CFU reduction under the same aBL exposure. NTHi 486 wt and ATCC 10211 exhibited 3.4-log₁₀ and 2.9-log₁₀ CFU reduction after an aBL exposure of 144 J/cm².

We selected 4 strains: NTHi RD118, NTHi 1003 wt, NTHi 375 wt, and ATCC 10211 for an in vitro study. In brief, overnight bacterial cultures were resuspended in BHI supplemented with 2 mg/mL nicotinamide adenine dinucleotide and 10 mg/mL hemin, to a concentration of 1-2×10⁸ CFU/mL. To form H. influenza biofilms, 200 μL of bacterial suspensions were placed in the wells of 96-well plates and incubated under 37° C. and ~5% CO₂ for 96 h. The culture medium was refreshed daily to avoid nutrient depletion.

Prior to aBL exposure, biofilms in the wells of 96-well plates were carefully washed two times with PBS and then overlaid with 100 μL of fresh PBS. Biofilms were exposed to increasing aBL exposures of 0, 54, 108, 162, and 216 J/cm². After treatment, biofilms were scraped from the wells using a sterile 200-μL pipette tip, suspended in 100 μL of PBS, and transferred to 1.5-mL microcentrifuge tubes. The process was repeated with 100 μL additional PBS. The samples collected in 1.5-mL microcentrifuge tubes were sonicated for 15 min using a Mechanical Ultrasonic Cleaner (e.g., M2800 available from Branson Ultrasonics) and then vortexed for 30 s. Bacterial viability was determined by CFU quantification, and the data was presented in log₁₀ CFU reduction.

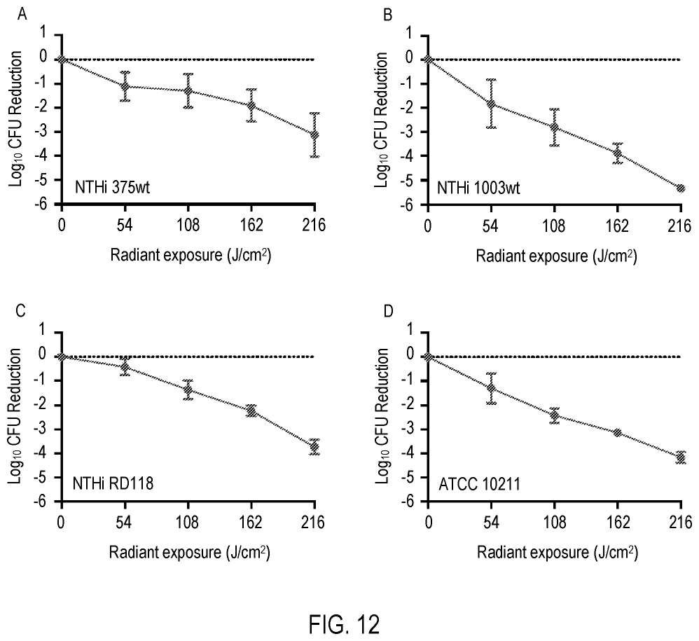

As shown in FIGS. 12A-D, aBL effectively killed H. influenzae in biofilms in a dose dependent manner. After an aBL exposure of 216 J/cm², 5.33-, 3.72-, and 3.12-log₁₀ CFU reduction was achieved in NTHi 1003 wt, NTHi RD118, and NTHi 375 wt, respectively.

To evaluate the biofilm-forming capability of different otopathogens on TTs, we selected two commercial collar button TTs made from silicone (e.g., VT-1001-01 available from Summit Medical) and fluoroplastic (e.g., VT-1001-01 available from Summit Medical), in addition to the Optical-TTs.

As in the above studies, aBL was delivered using a single 405 nm LED, which was positioned 4 cm above the outer facet of the TTs with the irradiance set to 60 mW/cm². The light energy emitted from the surface of the Optical-TTs was calculated by dividing the light energy received by the outer facet area (0.0611 cm²) of the TTs with the total surface area of the TTs (0.3911 cm²), taking into account the light loss during the transmission (~5%).

The otopathogens selected in this study included S. aureus (ATCC 25923 and ATCC 33592, AR 0215), P. aeruginosa (ATCC 27853, ATCC BAA-47, and AR0231), and H. influenzae (NTHi RD118, NTHi 1003 wt, and NTHi 375 wt). S. aureus and P. aeruginosa were routinely cultured on BHI agar and in BHI broth at 37° C. H. influenzae were cultured on Chocolate agar and in BHI broth supplemented with 2 mg/mL nicotinamide adenine dinucleotide and 10 mg/mL hemin, under 37° C. and ~5% CO2.

To assess the biofilm formation capability of otopathogens on TTs, TTs were placed in the wells of 96-well plates, each with 200 μL of bacterial suspension in fresh medium containing 1-2×10⁸ CFU/mL. The wells with S. aureus and P. aeruginosa cultures were incubated for 48 h, and those with H. influenzae cultures for 120 h. Growth media was refreshed daily. After the incubation, TTs with biofilms were carefully washed three times with PBS to remove planktonic cells.

After washing, TTs with biofilms were transferred to 1.5-mL Eppendorf tubes containing 50 μL of PBS+0.05% of Tween 80, sonicated in a Mechanical Ultrasonic Cleaner (e.g., M2800 available from Branson Ultrasonics) for 15 min to detach the biofilms on TTs, and then vortexed for 60 s. Bacterial suspensions in the Eppendorf tubes were serially diluted and plated on agar. CFU quantification was performed when CFUs are visible (18-24 h). All the experiments were performed in 3 independent replicates.

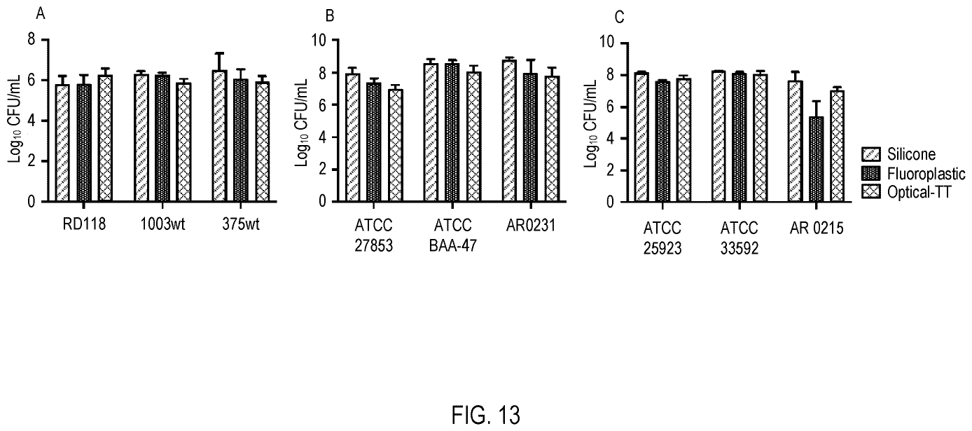

As seen in FIG. 13, all strains tested formed biofilms on TTs. For H. influenzae, ≥5.7-log₁₀ CFU/mL were detected in the biofilm on TTs. while for S. aureus (except AR 0215 strain) and P. aeruginosa, ≥7-log₁₀ CFU/mL was identified in the biofilms. The bacterial burden of S. aureus AR 0215 biofilms on fluoroplastic TT was found to be <5-log₁₀ CFU/mL.

After confirming the biofilm formation on the TTs by CFU quantification, we next evaluated the effectiveness of aBL in eradicating biofilms on Optical-TTs. Biofilms were formed on TTs as described above, washed three times in PBS, placed into the wells of 48-well plates, each containing 300 μL of BHI, and then exposed to aBL. aBL was delivered using a single 405 nm LED (e.g., M405L3 available from Thorlabs), which was positioned 4 cm above the plate surface, and the irradiance on the outer facet of the Optical TTs was set to 60 mW/cm². The light energy emitted from the surface of the Optical-TT was calculated by dividing the light energy received by the surface area of the outer facet (0.0611 cm²) with the total surface area of the Optical TT (0.3911 cm²), taking into account the light transmittance factor (95%),

The Optical-TTs were exposed to aBL at increasing radiant exposures of 0, 6, 12, 18, 24, and 30 J/cm² for H. influenzae and P. aeruginosa and 0, 12, 24, 36, 48, and 60 J/cm² for S. aureus. After aBL exposure, Optical-TTs were transferred to 1.5-mL microcentrifuge tubes, each containing 50 μL of PBS+0.05% of Tween 80, and sonicated for 15 min using a Mechanical Ultrasonic Cleaner (M2800; Branson Ultrasonics). The Eppendorf tubes containing the Optical-TTs were then vortexed for 1 min. Bacterial suspensions in the Eppendorf tubes were serially diluted and plated on agar. CFUs were quantified when they are visible (18-24 h after incubation) and the data was presented in logo CFU/mL reduction. All the experiments were performed in 3 independent replicates.

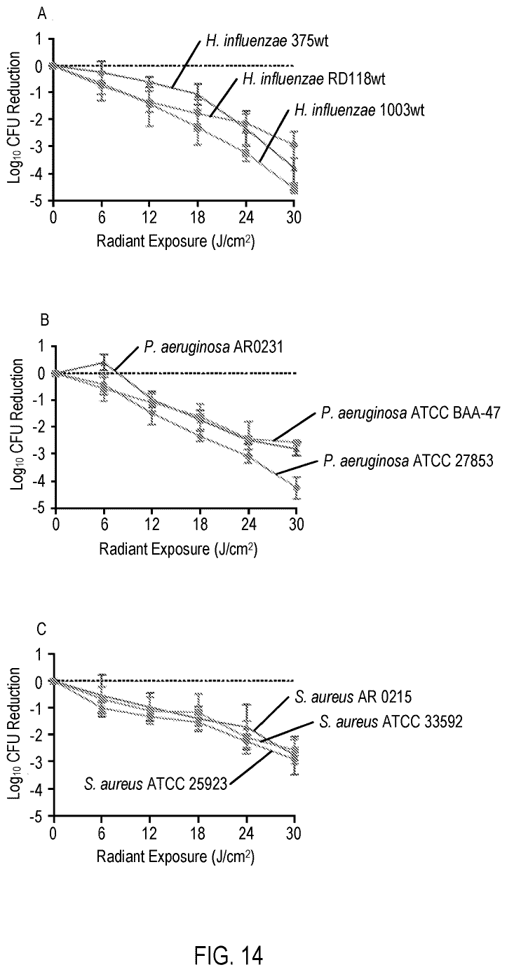

As shown in FIG. 14, aBL effectively killed otopathogens in biofilms on Optical-TTs produced by all tested bacterial strains. Over 2.5-log₁₀ CFU/mL reduction was achieved in H. influenzae and P. aeruginosa biofilms after only 30 J/cm² aBL and in S. aureus biofilms after 60 J/cm² aBL.

### Example 6: Experimental Tympanostomy Tube Otorrhea (TTO) in Chinchillas

Chinchillas weighing 500-650 g and 5-6 months old were used. To develop experimental TTO, an incision was made in the lower anterior quadrant of the tympanic membrane (TM) of chinchillas under general anesthesia, by using a speculum in the external auditory canal and under an operating microscope. Afterward, a TT was inserted at the site of the incision. Immediately following the placement of TTs, animal ears were inoculated with 100 μL of H. influenzae suspensions containing 10⁸ CFU through the TTs. Animals were then examined for the development of TTO by using daily tympanometry, otomicroscopy, and imaging. Once otorrhea developed, quantitative cultures were performed to confirm the disease. Subsequently, animals were followed daily, imaging was performed every 2-3 days. Around day 7-10, the bulla was excised and the TT was removed and examined under magnification for the presence of biofilm. Biofilm was observed on the TT removed from the eardrum.

To treat TTO in vivo, we have developed a prototype device for aBL delivery to the infection sites. The prototype contains an otoscope, which is used to monitor the light beam in real time and align the optics of the Optical TT in the eardrum. The device is also coupled with an auricular speculum and has a magnifying lens for eardrum examination. aBL is emitted from a diode 405 nm Fiber-Coupled Laser Module (#19-478, Edmund Optics), which is connected to the prototype. The laser output is fixed at 0.022 W and the size of light spot is ~5 mm in diameter.

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A system for preventing or treating tympanostomy tube infections, the system comprising:
an ear tube configured to be placed within an ear of a subject, the ear tube having a shaft extending from a proximal end to a distal end, wherein a lumen extends through the shaft, and having a flange extending from the shaft at the proximal end and a flange extending from the shaft at the distal end;
an optical waveguide configured to deliver antimicrobial light to a surface area of the ear tube while placed within the ear of the subject, the optical waveguide comprising an optical fiber configured to deliver the antimicrobial light and a fiber collimator configured to guide the antimicrobial light towards the surface area of the ear tube.

2. The system of claim 1, wherein the ear tube is fabricated out of a material with a light loss no greater than 0.2/mm for the antimicrobial light.

3. The system of claim 1, wherein the ear tube is fabricated from a primary material and further comprises a second material dispersed throughout the primary material, the second material being at least one of air bubbles, particles, or liquid drops, and the second material being configured to scatter the antimicrobial light with increased uniformity.

4. The system of claim 1, further comprising a moldable earplug, wherein an distal end of the optical fiber and a proximal end of the collimator are fixed within the earplug.

5. The system of claim 4, wherein the moldable earplug was custom-molded to fit into the ear of the subject.

6. The system of claim 1, further comprising a light source configured to emit antimicrobial light through the optical waveguide.

7. The system of claim 6, wherein the light source is one of a diode laser or a light-emitting diode (LED).

8. The system of claim 6, wherein the antimicrobial light has a wavelength between 405 nm and 420 nm.

9. The system of claim 6, wherein the antimicrobial light has a wavelength between 400 nm and 470 nm.

10. A method for treating or preventing tympanostomy tube infections, the method comprising the steps of:
(a) inserting a tympanostomy tube treatment system into an ear of a subject, the treatment system comprising a moldable earplug configured to rest in the ear of the subject and an optical waveguide configured to deliver antimicrobial light to a tympanostomy tube;
(b) delivering the antimicrobial light through the optical waveguide to a surface of the tympanostomy tube for a treatment duration.
(c) removing the tympanostomy tube treatment system from the ear of the subject.

11. The method of claim 10, wherein the antimicrobial light has a wavelength between 400 nm and 470 nm.

12. The method of claim 10, wherein the moldable earplug is further configured to align the optical waveguide with an outer surface of the tympanostomy tube.

13. An ear tube configured to be placed within a tympanic membrane of an ear of a subject, the ear tube comprising a shaft extending from a proximal end to a distal end, wherein a lumen extends through the shaft, and wherein the ear tube further comprises a first flange extending annularly from the shaft at the proximal end and a second flange extending annularly from the shaft at the distal end; and
wherein the ear tube is fabricated from a primary material sufficiently transparent to antimicrobial light, and wherein a plurality of pockets is dispersed throughout the primary material, the plurality of pockets being filled with a secondary material configured to scatter the antimicrobial light toward a surface of the ear tube.

14. The ear tube of claim 13, wherein the primary material is silicone.

15. The ear tube of claim 14, wherein the silicone comprises polydimethylsiloxane (PDMS) elastomer.

16. The ear tube of claim 13, wherein the primary material is polymethylmethacrylate (PMAA).

17. The ear tube of claim 13, wherein the ear tube was 3D printed.

18. The ear tube of claim 13, wherein the ear tube was constructed using a polymer molding technique.

19. The ear tube of claim 13, wherein the secondary material comprises at least one of a gas, a solid, or a liquid.

20. The ear tube of claim 13, wherein the secondary material comprises air.
